(19) United States
(12) Patent Application Publication
(10) Pub. No.: US 2010/0280393 A1
(43) Pub. Date: Nov. 4, 2010

(54) Optical Reflectance Spectroscopy for Evaluation of Radiation Injury

(75) Inventors: Tatiana G. Levitskaia, Kennewick, WA (US); James E. Morris, Richland, WA (US); Karla D. Thrall, West Richland, WA (US); Samuel A. Bryan, Richland, WA (US); James M. Peterson, West Richland, WA (US)

Correspondence Address: BATTELLE MEMORIAL INSTITUTE;ATTN: IP SERVICES, K1-53, P. O. BOX 999, RICHLAND, WA, 99352, US

(73) Assignee: BATTELLE MEMORIAL INSTITUTE, Richland, WA (US)

(21) Appl. No.: 12/772,530

(22) Filed: May 3, 2010

Related U.S. Application Data
(60) Provisional application No. 61/175,284, filed on May 4, 2009.

(51) Int. Cl. A61B 6/00; G06F 19/00

(52) U.S. Cl. 600/477; 702/19; 600/476; 356/445; 356/51

(57) ABSTRACT

Non-invasive methods and systems are described for rapidly measuring in-vivo dose, severity, and progression of injury after exposure to damaging phenomena, such as ionizing radiation, chemical burns, or electrical burns. Optical reflectance backscattering spectroscopy is applied to identify and characterize the effects of such phenomena on an individual's whole body and in localized areas.

### PRIORITY

This invention claims priority from currently pending U.S. provisional patent application No. 61/175,284, filed May 4, 2009, and entitled “Optical Reflectance Spectroscopy for Evaluation of Cutaneous Radiation Injury,” which is incorporated herein by reference.

### STATEMENT REGARDING FEDERALLY SPONSORED RESEARCH OR DEVELOPMENT

This invention was made with Government support under Contract DE-AC0576RLO1830 awarded by the U.S. Department of Energy. The Government has certain rights in the invention.

### BACKGROUND

Exposure to damaging phenomena such as chemical burns, electrical burns, or ionizing radiation from radiological sources can cause various health related disorders including death. The damage can occur deep within body tissues and may not be apparent for a variable, sometimes significant, period of time. If a rapid, reliable method were available, patients could be evaluated and triaged into various categories of trauma management, evaluation and treatment. For example, after a radiation incident, dosimetry assessment tools for rapid determination of exposure to ionizing radiation would be immensely helpful in screening, triage, and the management of clinical facilities in a mass casualty incident. Accordingly, what is needed is a method for making such determinations. The present invention provides for such a need.

Additional advantages and novel features of the present invention will be set forth as follows and will be readily apparent from the descriptions and demonstrations set forth herein. Accordingly, the following descriptions of the present invention should be seen as illustrative of the invention and not as limiting in any way.

### SUMMARY

The present invention is a non-invasive method and system for rapidly measuring in-vivo the dose, severity, and progression of injury after exposure to damaging phenomena, such as ionizing radiation, chemical burns, or electrical burns. Optical reflectance backscattering spectroscopy is applied to identify and characterize the effects of such phenomena on an individual's whole body and in localized areas. The invention provides means for population screenings, triage, early evaluations and potential intervention. This invention also allows for the estimation of the time of exposure. For example, this can be used for monitoring exposure to ionizing radiation from nuclear attacks, disasters, occupational, or cancer treatment, as well as monitoring for electrical or chemical burns. The invention can be utilized following either local or whole body exposure. The systems and methods of the present invention may also have applications for monitoring various other conditions involving tissue and vascular damage.

In one embodiment, dose, severity, and progression of whole body radiation exposure caused by ionizing radiation is characterized by acquiring in vivo spectral data using a non-invasive optical reflectance backscattering spectrometer over both visible and near-infrared regions of light. The measurements are taken through skin and subcutaneous tissue using, for example, a non-invasive optical probe connected to the spectrometer by fiber optics. Accordingly, the acquisition of the spectral data is not limited to the dermal and epidermal layers, but extends to subcutaneous and deeper tissue, blood, and circulation fluids. The invention further comprises analyzing the spectral data by performing multivariate analysis on appropriate programmed processing circuitry. Patterns and structures that represent whole body injury can then be identified in the analyzed data.

As used herein, ionizing radiation refers to electromagnetic waves or subatomic particles that have enough energy to remove electros from atoms and to break chemical bonds. Exemplary ionizing radiation includes, but is not limited to, gamma radiation, X rays, beta rays, alpha radiation, and neutrons.

In a preferred embodiment, the multivariate analysis comprises a multivariate pattern recognition analysis technique. Exemplary multivariate pattern recognition analysis techniques include, but are not limited to, principal component analysis (PCA), soft independent modeling of class analogy (SIMCA), and trend analysis. Such pattern recognition techniques are performed to automatically identify, or to assist in identifying, structures and patterns in the analyzed data that represent whole body injury.

In another embodiment, the multivariate analysis comprises linear or non-linear regression techniques, which are applied to provide for calibration and quantification of the analyzed data. Examples include, but are not limited to, classical least squares (CLS), evolving factor analysis (EFA), locally-weighted regression (LWR), multiple linear regression (MLR), multivariate analysis, partial least squares (PLS), and principal component regression (PCR). The calibration and quantification allows for the correlation of the analyzed data to received radiation dose, to a radiation injury severity measure, to time of exposure and kinetic progression of the radiation injury, and to a dose of an intervening agent administered as a treatment.

In some embodiments, prior to performing the multivariate analysis, the spectral data is preprocessed. Exemplary preprocessing techniques include, but are not limited to Derivative (Discrete Point and Savitzky/Golay), Integration, Kubelka-Munk correction, Multiplicative Scatter Correction, Polynomial subtraction, Scaling (Auto scaling, Mean Centering, Range scaling, and Variance scaling), Smoothing (Boxcar, Triangular, Gaussian, Savitzky-Golay), and Standard normal variate.

In preferred embodiments, the identification of structures in the analyzed data as well as the quantification or calibration of the analyzed data further comprises comparing the analyzed data to reference data obtained from a sample population. The reference data, for example, can eliminate the need for pre-exposure data and information from each individual. Rather, the post-exposure measurements from an individual can be compared to the reference data, which can provide the basis for identification, calibration, and quantification of the dose, severity, and progression of the radiation exposure.

In yet another embodiment, the in-vivo spectral data can be acquired over ultraviolet regions of light on a localized cutaneous region. After multivariate analysis of the localized spectral data, this would provide for identification of structures in analyzed data that represent localized cutaneous radiation injury in addition to the whole body information. As used herein, the cutaneous region refers to the dermis and epidermis layers.

The purpose of the foregoing abstract is to enable the United States Patent and Trademark Office and the public generally, especially the scientists, engineers, and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

Various advantages and novel features of the present invention are described herein and will become further readily apparent to those skilled in this art from the following detailed description. In the preceding and following descriptions, the various embodiments, including the preferred embodiments, have been shown and described. Included herein is a description of the best mode contemplated for carrying out the invention. As will be realized, the invention is capable of modification in various respects without departing from the invention. Accordingly, the drawings and description of the preferred embodiments set forth hereafter are to be regarded as illustrative in nature, and not as restrictive.

### DESCRIPTION OF DRAWINGS

Embodiments of the invention are described below with reference to the following accompanying drawings.

FIG. 1 shows spectra and analyzed data for naïve and Cs-137 injected animals according to embodiments of the present invention.

FIG. 2 shows optical absorption spectra before and after irradiation using embodiments of the present invention.

FIG. 3 shows analyzed data at various radiation doses according to embodiments of the present invention.

FIG. 4 shows analyzed data at various times post radiation exposure according to embodiments of the present invention.

### DETAILED DESCRIPTION

The following description includes the preferred best mode of one embodiment of the present invention. It will be clear from this description that the invention is not limited to these illustrated embodiments but that the invention also includes a variety of modifications and embodiments thereto. Therefore the present description should be seen as illustrative and not limiting. While the invention is susceptible of various modifications and alternative constructions, it should be understood, that there is no intention to limit the invention to the specific form disclosed, but, on the contrary, the invention is to cover all modifications, alternative constructions, and equivalents falling within the spirit and scope of the invention as defined in the claims.

The present invention aims to overcome many of the challenges facing field-friendly biodosimetry measurements by providing for quick identification of individuals exposed to external and/or internal ionizing radiation and estimation of the received dose and time of exposure. According to embodiments of the present invention, optical reflectance spectroscopy and multivariate data evaluation may offer the advantage of rapid high throughput non-invasive in vivo screening of potentially exposed populations. The present invention provides a low-cost method and system comprising an optical spectrometer coupled fiber optically with a reflectance/backscattering probe, which is placed against the skin. In use, light propagates through the epidermis/dermis skin layers and subcutaneous tissues in order to image potential changes in blood and deeper tissues due to whole body irradiation. The spectral data that is collected is subjected to a multivariate treatment, which can include, for example, a Principal Component Analysis (PCA) and Partial Least Square (PLS) modeling. Even in non-optimized configurations, in vivo (F344 rat model) measurements using off-the-shelf commercial instrumentation, embodiments of the present invention were able to detect changes in spectral signature utilizing the physiological response caused by radiation injury as biomarkers for whole-body exposure and to correlate them with the exposure dose.

Moderate- and high-dose radiation exposure results in the time-dependent pronounced changes in tissue, blood, and/or circulation fluids, likely due to the detection of alterations of coordination environment and oxidation state of iron in blood.

### EXAMPLE

Male F344 rats (125-170 g body weight) were obtained from Charles River Breeding Laboratory (Raleigh, N.C.). All animals were housed in solid-bottom cages with hardwood chip bedding and provided certified PMI 5002 Rodent Diet (Animal Specialties, Inc., Hubbard, Oreg.) and water ad libitum. Animals were acclimated in a humidity- and temperature-controlled room with a 12-h light/dark cycle for at least 3 days prior to use. The light cycle was 12 hour light/12 hour dark and relative humidity and temperature maintained at 50+15% and 22+2° C. All animal protocols were approved by the Institutional Animal Care and Use Committee at Pacific Northwest National Laboratory and studies were performed according to the “Guide for the Care and Use of Laboratory Animals” (National Research Council 1996). The animal facility at Battelle, Pacific Northwest Division is AAALAC accredited. For Cs-137 exposure, animals were purchased with indwelling jugular-vein cannula. Cannula patency was ensured by establishing that blood could be drawn into the cannula tubing, followed by flushing with heparinized saline using a disposable 1-mL syringe with a 23-gauge blunt-tip needle.

Animals were randomly assigned to one of 2 groups of n=5 animal per irradiation test groups. The whole body X-ray irradiation doses were chosen 3 and 6 Gy to ensure physiologically different responses. The X-ray radiation dose of 6.7 Gy was previously reported to be the LD50/30 for the rat model (see Mirjana et al., Shock 31(6):607-614, 2009). Accordingly the dose of 3 Gy is expected to induce non-lethal hematopoietic changes while the sub-lethal dose of 6 Gy is expected to result in hematopoietic/GI tract syndrome. Irradiated animals were housed singularly and monitored at days 1 through 9 post exposure in approximately 24 hour intervals using the optical spectroscopy system. To take a measurement, the optical probe was placed against the animal ear or shaved thigh; the probe was placed 1 mm above the surface of the skin Each measurement was acquired in triplicate. Measurements taken from non-irradiated animals served as control. To confirm that spectral changes observed for irradiated vs control animals was due to the internal physiological changes rather than potential cutaneous damage, additional group of animals (n=10) was given whole body irradiation dose (not calculated) using Cs-137 internal exposure. Solution of Cs-137 was intravenously injected, and animals were optically measured 24 hr post exposure. The administration of the radionuclide by the IV route was purposely selected to model whole body irradiation via systemic circulation of radioactivity, without delays in absorption as might be observed with dermal, oral or inhalation exposures. Spectral measurements taken prior to whole body external or Cs-137 exposure served as controls.

All animals were whole-body X-ray irradiated using a Therapax X-RAD 320 system equipped with 320 kV high stability X-ray generator, metal ceramic X-ray tube, variable X-beam collimator and #8 filter (Precision X-ray Incorporated, East Haven, Conn.). Radiation doses of 3 and 6 Gy were achieved with irradiator settings at 0.76 Gy/min at 300 kV for 4 and 8 min, respectively. Internally irradiated animals received a single IV injection (0.2 mL) of Cs-137 as a chloride salt in a buffered sterile saline solution at a dose of 6.8±0.2 kBq via the indwelling jugular vein cannula.

Visible-near Infrared (vis-NIR) spectrophotometric measurements were performed using 400-series charge-coupled device array spectrometer (Spectral Instruments Inc.), 350-950 nm scan range and Ocean Optics NIR-512 spectrometer, 850-2500 nm scan range coupled with LS-1 tungsten halogen light source and 400 series dual-source reflectance-backscattering probe (SI Photonics). The STAN-SSH High-reflectivity Specular Reflectance Standard (Ocean Optics) was used to reference the probe. The optical probe was placed against an animal's ear, and spectra were collected in triplicate from the right and left ears.

The principal component analysis (PCA) and partial least squares (PLS) functions and algorithms were performed using Matlab Statistics Toolbox (Mathworks Inc., USA) and DeLight Chemometrics Software (DSquared Inc., USA). Initial analysis of the spectral data sets involved PCA treatment, which allowed for exploration of the data to give general information regarding initial models and type of data preprocessing to be used. Typical preprocessing of spectra included curve smoothing, baseline normalization, and performing a first or second derivative of the spectra. The goal of PCA analysis was to represent the variation present in the spectral data using a small number of factors, or principal components (PCs). After data reduction to the principal components, human pattern recognition was used to identify structures within the data. Significant optical data variability was observed due to several external factors including movement of unrestricted animal, usage of the non-optimized instrumentation leading to the variable pressure applied by the operator to animal skin, ambient background light, location of the optical collection, and other. To identify outliers within each of the data sets, a 95% probability ellipsoid was computed for each subset indicating that data within each ellipsoid has a 95% probability of residing within that group. The data points located outside the 95% probability ellipsoid were excluded from the further analysis.

The goal of chemometric PLS modeling is to correlate the measured optical response to the dose delivered to each individual animal and time post-exposure. In the PLS analysis, the concentration (dose) vector is treated as a function of the response matrix. In this treatment, the response derived from PCA treatment as providing a more robust computation was used.

Both visible and NIR spectral regions are of critical importance to the measurements. Visible region is responsive to the changes associated with colored components of the blood, most notably Fe(II)-Fe(III) and its chelates with iron-binding proteins that are sensitive to the ionizing radiation exposure.

Studies in which whole body irradiation was induced by internal Cs-137 indicated that the measured spectral shifts were due to internal biochemistry rather than localized changes to the cutaneous layers. It is understood that whole-body exposure to moderate and high doses of ionizing radiation introduces multiple physiological and biochemical changes in the body. The complexity of these changes can be detected and related to the received dose and time of exposure using relatively simple spectrophotometric measurements.

In hemoglobin, the iron ion may be either in the Fe(II) or Fe(III) state. When blood cells bind oxygen, Fe(II) oxidizes to Fe(III); once in the 3+ oxidation state, the binding site can no longer bind oxygen. This process occurs naturally in the body. The enzyme methemoglobin reductase reactivates hemoglobin found in the Fe(III) state by reducing the iron center. If radiation-induced oxidative stress can cause this conversion in iron and other metals, then many catalytic functions in the body could be disrupted. Evidence of this disruption by radiation was found in blood including alteration in structure and function of hemoglobin. Radiation can also induce changes in iron binding by blood proteins. Levels of non-transferrin bound iron have been shown to increase following whole body irradiation causing toxicity.

Due to high absorptivity of water in NIR regions and the absorptions associated with overtones and combination bands of the fundamental molecular vibrations of OH, NH and CH functional groups typical for peptides and aminoacids, variations in the circulation fluids upon ionizing radiation exposure were observed in the NIR spectral region. NIR spectral region was found to be free of optical interferences due to the skin components absorbing light in the UV and visible regions, e.g. melanin, and thus insensitive to their variation in different skin types.

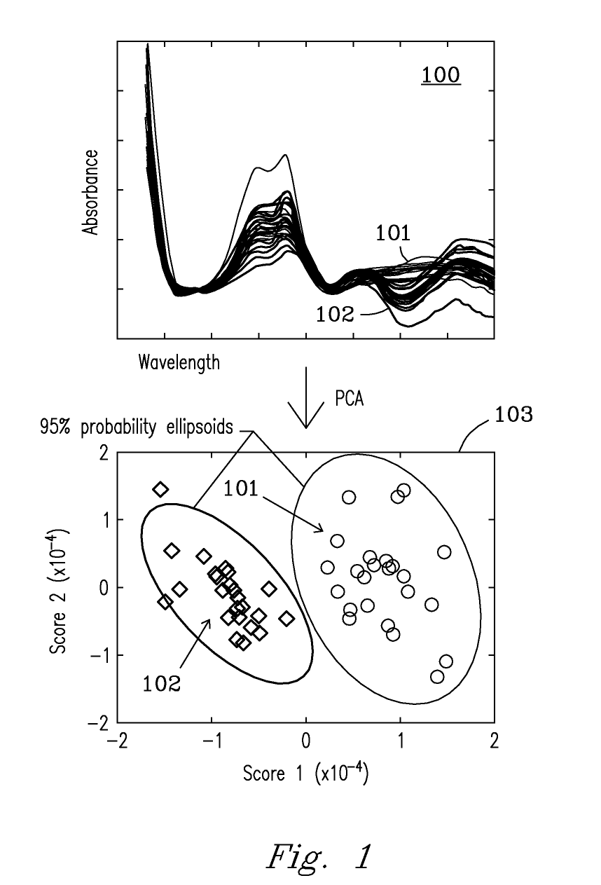

FIGS. 1-4 depict a variety of aspects and embodiments of the present invention. Referring first to FIG. 1, spectra 100 of 16 animals, 8 naïve 101 and 8 exposed 102, in triplicate, for a total of 48 measurements, and a PCA scores plot 103 of the spectroscopic data of naïve and Cs-137 injected animals at 24 hrs post radionuclide exposure are shown. Whole gamma irradiation of F344 rats was produced via internal exposure to Cs-137 (intravenous injection, 6.8±0.2 kBq). Optical measurements were made 24 hours following exposure, and PCA treatment delineated the radiated (Cs-137) and non-radiated animals. This result indicates that the measured spectral shifts were due to internal biochemistry rather than localized changes to the cutaneous layers.

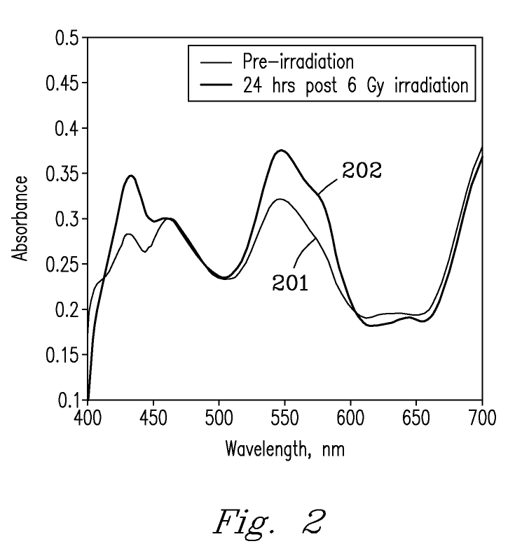

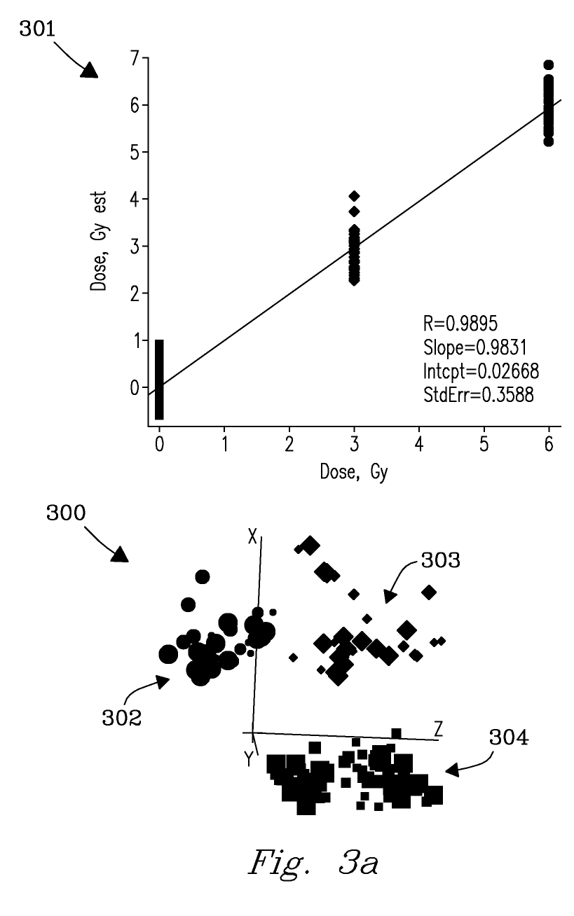

FIG. 2 shows optical absorption spectra that depict a comparison of the representative optical absorption spectra acquired using reflectance probe placed against an ear of an individual F344 rat before and 24 hrs after 6 Gy X-ray whole body irradiation, 201 and 202, respectively. It is significant that spectral changes are measurable shortly after irradiation.

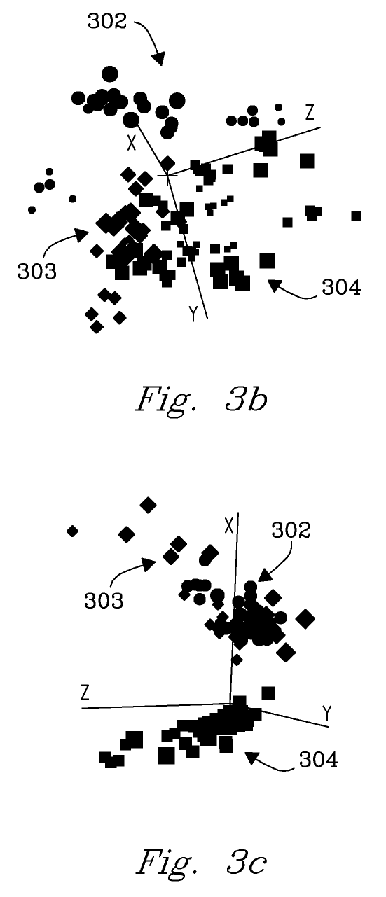

FIGS. 3a-3c show PCA differentiation and PLS prediction (R²=0.989) of radiation dose using optical reflectance measurements of F344 rats exposed to whole body X ray irradiation (0, 3, 6 Gy, n=10) at 24 hours post radiation exposure (304, 303, and 302, respectively). The PCA plot 300 in FIG. 3a shows results of spectral measurements collected using animals prior irradiation (green) as well as animals exposed to 3 Gy (red) and 6 Gy (blue) X-ray whole body irradiation. The PLS plot 301 estimates dose of exposure. Chemometric treatment was done using both visible 420-750 nm and NIR 900-1500 nm spectral regions (FIG. 3a). For comparison, PCA plots obtained using visible only (FIG. 3b) and NIR only (FIG. 3c) regions are shown.

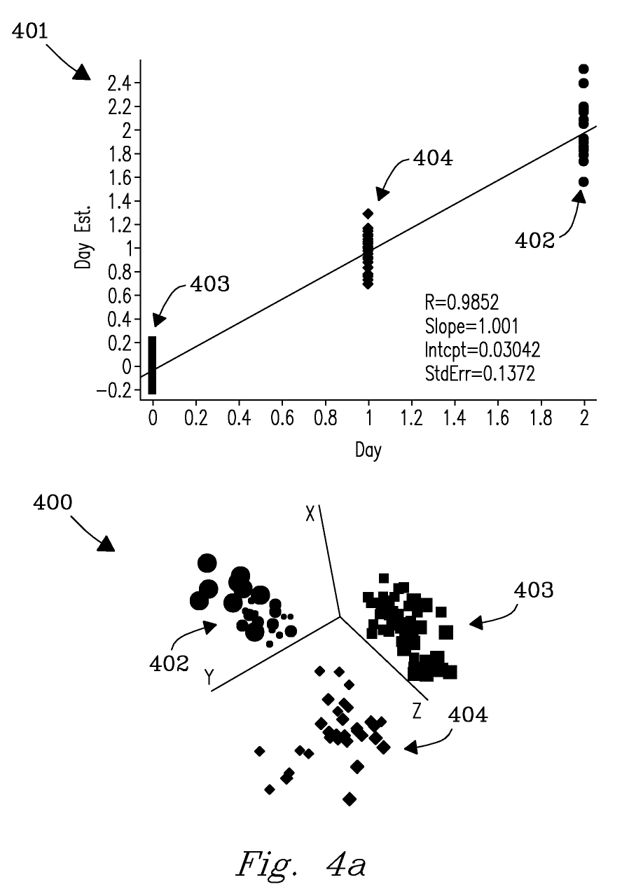

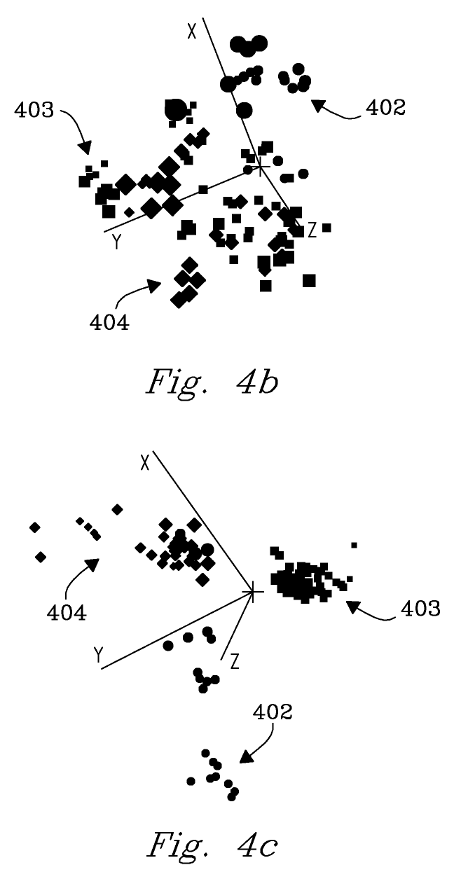

FIGS. 4a-4c show PCA differentiation and PLS prediction (R²=0.985) of time post radiation exposure using optical reflectance measurements of F344 rats (n=10). The PCA plot 400 (FIG. 4a) shows pre-irradiation results collected prior to irradiation 403 as well as 24 hrs 404 and 48 hrs 402 following 3 Gy X-ray whole body irradiation. The PLS plot 401 (FIG. 4a) estimates time post exposure (0, 24 and 48 hours). Chemometric treatment was done using both visible 420-750 nm and NIR 900-1500 nm spectral regions. For comparison, PCA plots obtained using visible only (FIG. 4b) and NIR only (FIG. 4c) regions are shown.

While a number of embodiments of the present invention have been shown and described, it will be apparent to those skilled in the art that many changes and modifications may be made without departing from the invention in its broader aspects. The appended claims, therefore, are intended to cover all such changes and modifications as they fall within the true spirit and scope of the invention.

We claim:

1. A method for ascertaining dose, severity, and progression of whole body radiation exposure caused by ionizing radiation, the method characterized by:
acquiring in-vivo spectral data by non-invasive optical reflectance backscattering spectroscopy over both visible and near-infrared regions of light through skin and subcutaneous tissue;
performing multivariate analysis on the spectral data, thereby resulting in analyzed data; and
identifying structures in the analyzed data that represent whole body injury resulting from ionizing radiation exposure.

2. The method as recited in claim 1, wherein said identifying further comprises comparing the analyzed data to reference data from a sample population.

3. The method as recited in claim 1, wherein said acquiring in-vivo spectral data further comprises non-invasive optical reflectance backscattering spectroscopy over ultraviolet regions of light on a localized cutaneous region.

4. The method as recited in claim 3, further comprising identifying structures in the analyzed data that represent localized cutaneous radiation injury.

5. The method as recited in claim 1, wherein the multivariate analysis comprises multivariate pattern recognition analysis techniques.

6. The method as recited in claim 1, wherein the multivariate analysis comprises multivariate linear or non-linear regression techniques.

7. The method as recited in claim 6, further comprising correlating the analyzed data to received radiation dose.

8. The method as recited in claim 6, further comprising correlating the analyzed data to a radiation injury severity measure.

9. The method as recited in claim 6, further comprising correlating the analyzed data to time of exposure and kinetic progression of the radiation injury.

10. The method as recited in claim 6, further comprising administering a dose of an intervening agent as treatment for radiation injury, wherein the dose of the intervening agent correlates to the analyzed data.

11. The method as recited in claim 1, wherein said acquiring further comprises, detecting alterations to coordination environment, oxidation state, or both of iron in blood.

12. A system for ascertaining dose, severity, and progression of whole body radiation exposure caused by ionizing radiation, the system characterized by:
an optical reflectance backscattering spectrometer acquiring in-vivo spectral data over both visible and near IR regions of light through skin and subcutaneous tissue using a non-invasive optical probe;
processing circuitry programmed to perform multivariate analysis on the spectral data, thereby resulting in analyzed data, and to identify structures in the analyzed data that represent whole body injury.

13. The system as recited in claim 12, further comprising reference data from a sample population stored in a memory device to which analyzed data can be compared.

14. The system as recited in claim 12, wherein the optical reflectance backscattering spectrometer further acquires spectral data over ultraviolet regions of light on a localized cutaneous region.

15. The system as recited in claim 14, wherein the processing circuitry is further programmed to identify structures in the analyzed data that represent injury at the localized cutaneous region.

16. The system as recited in claim 12, wherein the multivariate analysis comprises multivariate pattern recognition analysis techniques.

17. The system as recited in claim 12, wherein the multivariate analysis comprises multivariate linear or non-linear regression techniques.

18. The system as recited in claim 17, wherein the processing circuitry is further programmed to correlate the analyzed data to received radiation dose.

19. The system as recited in claim 17, wherein the processing circuitry is further programmed to correlate the analyzed data to time of exposure and kinetic progression of radiation injury.

20. The system as recited in claim 17, wherein the processing circuitry is further programmed to correlate the analyzed data to a radiation injury severity measure.

21. The system as recited in claim 17, wherein the processing circuitry is further programmed to correlate the analyzed data to a dose of an intervening agent administered as treatment for radiation injury.
